(19) United States
(12) Patent Application Publication
(10) Pub. No.: US 2006/0142574 A1
(43) Pub. Date: Jun. 29, 2006

(54) Process for the preparation of ganciclovir

(76) Inventors: Jayachandra Suresh Babu, Haryana (IN); Purna Chandra Ray, Delhi (IN); Chandra Has Khanduri, Haryana (IN); Yatendra Kumar, Haryana (IN)

Correspondence Address: RANBAXY INC., 600 COLLEGE ROAD EAST, SUITE 2100, PRINCETON, NJ, 08540, US

(21) Appl. No.: 10/532,910

(22) PCT Filed: Oct. 31, 2003
(86) PCT No.: PCT/IB03/04866

§ 371 Date: Jan. 31, 2006

(30) Foreign Application Priority Data
Oct. 31, 2002 (IN) 1097/DEL/2002

(51) Int. Cl. C07D 473/10

(52) U.S. Cl. 544/276

(57) ABSTRACT

The present invention relates to a process for the preparation of N2-Acetyl-9-(1,3-diace-toxy-2-propoxymethyl) guanine, referred to here as N-9 alkylated isomer of structural formula I, and to the use of this compound as an intermediate for the preparation of antiviral compound, ganciclovir.

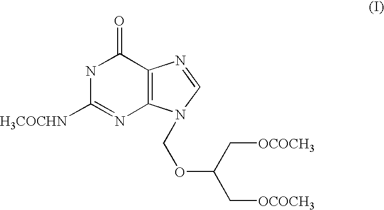

### FIELD OF THE INVENTION

The field of the invention relates to a process for the preparation of N²-Acetyl-9-(1,3-diacetoxy-2-propoxymethyl) guanine, referred to here as N-9 alkylated isomer of structural Formula I, and to the use of this compound as an intermediate for the preparation of antiviral compound, ganciclovir.

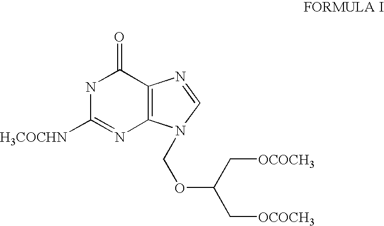

### BACKGROUND OF THE INVENTION

Chemically, ganciclovir is 9-(1,3-dihydroxy-2-propoxymethyl)guanine of structural Formula II, 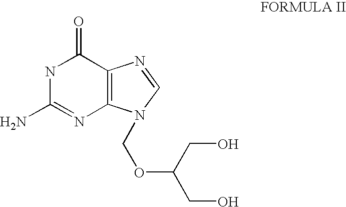
and is known from U.S. Pat. No. 4,355,032. It is one of the most important acyclic nucleosides having significant antiviral properties. It is highly efficacious against viruses of the herpes family and cytomegalovirus.

A number of methods are reported in the literature for the production of acylic purine nucleosides such as acyclovir and ganciclovir for example, methods which use guanine, diacetyl guanine, 2,6-dichloropurine, 2-amino-6-chloropurine (see U.S. Pat. No. 4,146,715 to Schaeffer); tetraacetylguanosine (J. Boryski et. al., Nucleosides and Nucleotides, 1989, 8, 529); acetylguanine (Japanese Patent Application No. 84-80685) or 2-chloro-6-iodopurine (J. R. Barrio et al., J Med. Chem., 1980, 23, 572) as starting materials.

The simplest synthetic approach to the N-9 substituted guanine compounds involves the direct alkylation of appropriately substituted 2-aminopurines, for example guanine derivatives which on deprotection of the functional group are converted to final products.

During the course of the present investigation, it has been discovered that diacetyl guanine (DAG) of structural Formula III, the starting material for preparing ganciclovir maybe converted to monoacetyl guanine (MAG) of structural Formula IV, some of which remains unreacted during the condensation reaction with 2-acetoxymethoxy-1,3-diacetoxy propane to give the N-9 alkylated isomer of structural Formula I.

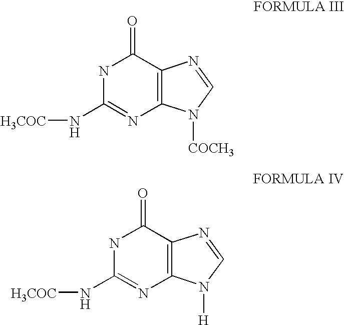

There are significant drawbacks to this approach as the penultimate intermediate i.e. N-9 alkylated isomer so produced is always accompanied with certain impurities, such as:
(a) unreacted starting material i.e. diacetyl or monoacetyl guanine.
(b) N²-Acetyl-7-(1,3-diacetoxy-2-propoxymethyl) guanine, referred to as N-7 isomer of structural Formula V, and 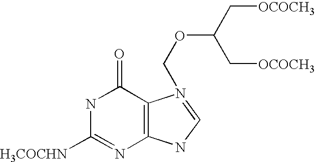
(c) some polar impurities

The prior art approach is not suitable from commercial point of view because the desired N-9 isomer requires purification by tedious and cumbersome purification processes such as column chromatography, HPLC or other techniques, thus making the approach commercially difficult to implement.

To achieve a high efficiency of reaction for industrial scale synthesis of ganciclovir, it is necessary to minimize the unreacted diacetyl or monoacetyl guanine, N-7 isomer and polar impurities.

Thus, the present invention provides a process which does not require the purification of the penultimate intermediate or the final product by HPLC or other techniques, rather uses organic solvents and/or water or mixtures thereof. The choice of which has been found to be important for removing the traces of polar and non- polar impurities.

### SUMMARY OF THE INVENTION

In one general aspect there is provided a process for the preparation of N²-acetyl-9-(1,3-diacetoxy-2-propoxymethyl) guanine, the N-9 alkylated isomer in pure form. The process includes obtaining a solution of N²-acetyl-9-(1,3-diacetoxy-2-propoxymethyl) guanine in one or more solvents; and recovering the pure N²-acetyl-9-(1,3-diacetoxy-2-propoxymethyl) guanine by the removal of the solvent.

The solvent may be one or more of lower alkanol, ketone, chlorinated solvent, water, or mixtures thereof. The lower alkanol may include one or more of primary, secondary and tertiary alcohol having from one to six carbon atoms. The lower alkanol may include one or more of methanol, ethanol, denatured spirit, n-propanol, isopropanol, n-butanol, isobutanol and t-butanol. In particular, the lower alkanol may include one or more of methanol, ethanol, and denatured spirit.

The ketone may include one or more of acetone, 2-butanone, and 4-methylpentan-2-one. The chlorinated solvent may include one or more of dichloromethane, dichloroethane and chloroform. Removing the solvent may include one or more of distillation, distillation under vacuum, filtration, filtration under vacuum, decantation and centrifugation.

The process may include further drying of the product obtained.

In one general aspect, the solution containing N²-acetyl-9-(1,3-diacetoxy-2-propoxymethyl) guanine may be cooled and filtered to remove unreacted solids before the removal of the solvent.

In another general aspect additional solvent may be added to residue obtained after removal of the solvent and it may be cooled before filtration to obtain better yields of the N-9 isomer.

The pure N-9 isomer has a purity of more than 98% having less than about 0.5% of monoacetyl and diacetyl impurity and less than about 0.5% of N-7 alkylated isomer impurity. More particularly, the purity of the N-9 isomer is more than 98.5% having less than about 0.15% of monoacetyl and diacetyl impurity and less than about 0.15% of N-7 alkylated isomer impurity.

In another general aspect there is provided a process for the preparation of ganciclovir by hydrolysis of the purified N-9 isomer.

The details of one or more embodiments of the inventions are set forth in the description below. Other features, objects and advantages of the inventions will be apparent from the description and claims.

### DETAILED DESCRIPTION OF THE INVENTION

The inventors have developed an efficient process for the preparation of N²-acetyl-9-(1,3-diacetoxy-2-propoxymethyl) guanine in pure form, by treating the N-9 alkylated isomer with one or more of solvents and recovering the pure N-9 isomer by the removal of the solvent.

In general, the solution of N²-acetyl-9-(1,3-diacetoxy-2-propoxymethyl) guanine may be obtained by dissolving N²-acetyl-9-(1,3-diacetoxy-2-propoxymethyl) guanine in a suitable solvent. Alternatively, such a solution may be obtained directly from a reaction in which N²-acetyl-9-(1,3-diacetoxy-2-propoxymethyl) guanine is formed. The solvent may be removed from the solution by a technique which includes, for example, distillation, distillation under vacuum, filtration, filtration under vacuum, decantation, and centrifugation.

The term “suitable solvent” includes any solvent or solvent mixture in which N-9 alkylated isomer is soluble, including, for example, lower alkanol, ketones, chlorinated solvents, water and mixtures thereof. Examples of alkanol include those primary, secondary and tertiary alcohols having from one to six carbon atoms. Suitable lower alkanol solvents include methanol, ethanol, denatured spirit, n-propanol, isopropanol, n-butanol, isobutanol and t-butanol. Examples of ketones include solvents such as acetone, 2-butanone, and 4-methylpentan-2-one. A suitable chlorinated solvent includes one or more of dichloromethane, dichloroethane and chloroform. Mixtures of all of these solvents are also contemplated.

In one aspect, the solution containing N²-acetyl-9-(1,3-diacetoxy-2-propoxymethyl) guanine can be cooled followed by filtration to remove any unreacted solids before the removal of the solvent.

In another aspect, additional solvent can be added to residue obtained after removal of the solvent and it can be cooled before filtration.

The product obtained may be further or additionally dried to achieve the desired moisture values. For example, the product may be further or additionally dried in a tray drier, dried under vacuum and/or in a Fluid Bed Drier.

Methods known in the art may be used with the process of this invention to enhance any aspect of this invention. For example, the solution containing the mixture of N-7 and N-9 isomers may be heated for dissolution, or may be cooled to separate out the product or the slurry may further be cooled prior to filtration or the solution may be seeded with seed crystals of the product to enhance precipitation of the product.

The N²-Acetyl-9-(1,3-diacetoxy-2-propoxymethyl) guanine so obtained may be hydrolyzed to give ganciclovir by the methods known in the literature (J. E. Martin et. al. J. Med. Chem., 1983,26,759-761).

The present invention is further illustrated by the following examples which are provided merely to be exemplary of the inventions and is not intended to limit the scope of the invention. Certain modifications and equivalents will be apparent to those skilled in the art and are intended to be included within the scope of the present invention.

### EXAMPLE 1

Crude N²-Acetyl-9-(1,3-diacetoxy-2-propoxymethyl)guanine (100 kg) was added to the mixture of dichloromethane (500 lit) and methanol (40 lit). Temperature was raised to 30-35° C. and maintained for 30 minutes and then activated carbon (5 kg) was added and stirred for another 30 minutes at the same temperature. Slowly cooled to 5° C. and maintained for 30 minutes. Filtered through celite bed, removed the solvent completely by distillation, added acetone (800 lit.) to the resulting mass. Cooled to 35° C., stirred for 60 minutes at 30-35° C. Filtered the solids and washed with acetone, yielding 80-82 kg of pure N²-Acetyl -9-(1,3-diacetoxy-2-propoxymethyl) guanine.

| Data on | Before | After |
|---|---|---|
| chromatographic purity | Purification | Purification |
| N-9 isomer | 95.08 | 98.90 |
| DAG/MAG | 2.77 | 0.1 |
| N-7 isomer | 0.62 | 0.11 |

### EXAMPLE 2

Crude N²-Acetyl -9-(1,3-diacetoxy-2-propoxymethyl) guanine (100 gm) was added to the mixture of dichloromethane (500 ml) and methanol (40 ml). Temperature was raised to 30-35° C. and kept for 30 minutes, and then activated carbon (5 gm) was added and stirred for another 30 minutes at the same temperature. Slowly cooled to 8° C. and maintained for 30 minutes. Filtered through celite bed and washed the bed using dichloromethane. Solvent was completely distilled off under vacuum. Charged fresh dichloromethane (200 ml) and heated up to 40° C. followed by cooling to 2-5° C. Filtered the product and washed with dichloromethane. Collected the wet material and charged acetone (700 ml) to the wet mass and heated to reflux temperature. Cooled to 35° C. stirred 60 minutes at 30-35° C. Filtered the solids and washed with acetone, yielding 68-72 gm of pure N²-Acetyl -9-(1,3-diacetoxy-2-propoxymethyl) guanine after drying.

| Data on | Before | After |
|---|---|---|
| chromatographic purity | Purification | Purification |
| N-9 isomer | 88.73 | 98.63 |
| DAG/MAG | 7.9 | 0.31 |
| N-7 isomer | 1.08 | 0.25 |

### EXAMPLE 3

Crude N²-Acetyl -9-(1,3-diacetoxy-2-propoxymethyl)guanine (100 gm) was added to the DM water (250 ml.) at room temperature. Temperature was raised to 70-75° C. and kept 30 minutes, all solids completely dissolved at the same temperature. Slowly cooled to room temperature followed by further cooling to 5-10° C. and maintained for 60 minutes. Filtered the product at 5° C., yielding 58 gm of pure N²-Acetyl-9-(1,3-diacetoxy-2-propoxymethyl)guanine after drying.

| Data on | Before | After |
|---|---|---|
| chromatographic purity | Purification | Purification |
| N-9 isomer | 77.15 | 86.73 |
| DAG/MAG | 3.01 | 1.65 |
| N-7 isomer | 13.64 | 6.64 |

### EXAMPLE 4

Crude N²-Acetyl -9-(1,3-diacetoxy-2-propoxymethyl)guanine (400 gm) was added to methanol (1.25 lit.) at room temperature. Temperature was raised to 40-45° C. and kept for 30 minutes, all solids completely dissolved at the same temperature. Slowly cooled to room temperature followed by further cooling to 5° C. and maintained for 60 minutes. Filtered the product at 5° C. and washed using chilled methanol, yielding 220 gm of pure N²-Acetyl -9-(1,3-diacetoxy-2-propoxymethyl)guanine after drying.

| Data on | Before | After |
|---|---|---|
| chromatographic purity | Purification | Purification |
| N-9 isomer | 88.41 | 94.09 |
| DAG/MAG | 1.71 | 0.34 |
| N-7 isomer | 4.83 | 1.06 |

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A process for the preparation of N²-Acetyl-9-(1,3-diacetoxy-2-propoxymethyl)guanine of Formula I in pure form, the process comprising:
obtaining a solution of N²-Acetyl-9-(1,3-diacetoxy-2-propoxymethyl) guanine in one or more solvents; and recovering the N²-Acetyl-9-(1,3-diacetoxy-2-propoxymethyl) guanine in pure form by the removal of the solvent.

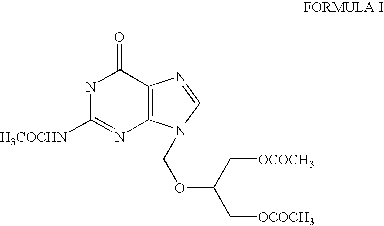

2. The process of claim 1, wherein the solvent comprises one or more of lower alkanol, ketone, chlorinated solvent, water, or mixtures thereof.

3. The process of claim 2, wherein the lower alkanol comprises one or more of primary, secondary and tertiary alcohols having from one to six carbon atoms.

4. The process of claim 2, wherein the lower alkanol comprises one or more of methanol, ethanol, denatured spirit, n-propanol, isopropanol, n-butanol, isobutanol, and t-butanol.

5. The process of claim 2, wherein the lower alkanol comprises one or more of methanol, ethanol, and denatured spirit.

6. The process of claim 2, wherein the ketone comprises one or more of acetone, 2-butanone, and 4-methylpentan-2-one.

7. The process of claim 2, wherein the chlorinated solvent comprises one or more of chloroform, dichloromethane and dichloroethane.

8. The process of claim 1, wherein removing the solvent comprises one or more of distillation, distillation under vacuum, filtration, filtration under vacuum, decantation, and centrifugation.

9. The process of claim 1, further comprising additional drying of the product obtained.

10. The process of claim 1, further comprising cooling the solution and filtering unreacted solids before removal of the solvent.

11. The process of claim 10, further comprising adding additional solvent after removal of the solvent and cooling.

12. The process of claim 11, further comprising the removal of the solvent.

13. The process of claim 1, wherein the N²-Acetyl-9-(1,3-diacetoxy-2-propoxymethyl) guanine obtained has a purity of more than 98%.

14. The process of claim 13, wherein the N²-Acetyl-9-(1,3-diacetoxy-2-propoxymethyl) guanine obtained has a purity of more than 98.5%.

15. The process of claim 14, wherein the N²-Acetyl-9-(1,3-diacetoxy-2-propoxymethyl) guanine obtained has a purity of more than 98.8%.

16. The process of claim 1, wherein the N²-Acetyl-9-(1,3-diacetoxy-2-propoxymethyl) guanine obtained has less than 0.5% of monoacetyl and diacetyl guanine impurity.

17. The process of claim 1, wherein the N²-Acetyl-9-(1,3-diacetoxy-2-propoxymethyl) guanine obtained has less than 0.15% of monoacetyl and diacetyl guanine impurity.

18. N²-Acetyl-9-(1,3-diacetoxy-2-propoxymethyl)guanine having a purity of more than 98%.

19. N²-Acetyl-9-(1,3-diacetoxy-2-propoxymethyl)guanine having a purity of more than 98.5%.

20. N²-Acetyl-9-(1,3-diacetoxy-2-propoxymethyl)guanine having a purity of more than 98.8%.

21. N²-Acetyl-9-(1,3-diacetoxy-2-propoxymethyl)guanine containing less than 0.5% of monoacetyl and diacetyl guanine impurity.

22. N²-Acetyl-9-(1,3-diacetoxy-2-propoxymethyl)guanine having less than 0.15% of monoacetyl and diacetyl guanine impurity.

23. A process for the preparation of ganciclovir of Formula II, the process comprising hydrolyzing N²-Acetyl-9-(1,3-diacetoxy-2-propoxymethyl) guanine of formula I prepared by the process of claim 1. [chemical structure]
